(19) United States
(12) Patent Application Publication
(10) Pub. No.: US 2020/0038139 A1
(43) Pub. Date: Feb. 6, 2020

(54) DENTAL TREATMENT INSTRUMENT FOR OPERATING A ROTATIONAL TOOL

(71) Applicant: KALTENBACH & VOIGT GMBH, Biberach (DE)

(72) Inventors: Jürgen BUTSCHER, Leutkirch (DE); Thomas CLASSEN, Herbertingen (DE)

(21) Appl. No.: 16/337,723

(22) PCT Filed: Sep. 28, 2017
(86) PCT No.: PCT/EP2017/074651

§ 371 Date: Mar. 28, 2019

(30) Foreign Application Priority Data
Sep. 28, 2016 (EP) 16191082.3

(51) Int. Cl. A61C 1/14; A61B 17/16

(52) U.S. Cl. CPC A61C 1/144; A61B 17/162

(57) ABSTRACT

The invention relates to a dental treatment instrument for operating a rotational tool. The treatment instrument has a head drive, which is provided for coupling to a shaft of the rotational tool and comprises a sleeve-like guide bushing (1), and a collet (9) for fixing the rotational tool. The guide bushing (1) has surface sections (3) which form parts of a cylindrical shape in at least one first axial region. Recesses (4) are provided which face radially outwards from the surface sections (3) and which impart a shape deviating from a rotationally symmetrical shape to the inner contour of the guide bushing (1). By virtue of the surface sections (3), a circular cylindrical shaft of a rotational tool can be guided or supported in a particularly suitable manner. The recesses (4) are additionally suitable for engaging radial protrusions of a non-circular cylindrical shaft of a rotational tool such that the non-circular cylindrical shaft can be driven in a particularly suitable manner. The treatment instrument according to the invention thus offers the possibility of receiving tool shafts with a correspondingly modified shaft geometry in order to meet particularly high corresponding requirements and counteract the aforementioned disadvantages and weaknesses of a shaft according to DIN EN ISO 1797 and the possibility of additionally using the tools according to DIN EN ISO 1797 for standard applications.

The invention relates to a dental treatment instrument for operating a rotary tool.

From the prior art, it is known to remove a (natural or artificial) dental crown using a dental treatment instrument and a rotary tool. In this case, it is generally desirable within the field of dental practice to keep the time required for this as short as possible. To enable a corresponding operating procedure to be suitably realized, there are various tools on the tool market which are matched to the different material properties of artificial crowns. The crowns are divided into the following types:
Metal crown (gold crown, titanium or steel)
Veneer crown (metal framework, ceramic veneer)
Full ceramic crown (zircon, feldspar, glass or aluminum oxide ceramics)
Synthetic crown

The rotary tools differ here in terms of the cutting geometries matched to the respective substance, the materials used and the coatings. The known rotary tools all have the, as such, conventional cylindrical shaft according to standard DIN EN ISO 1797 (in particular DIN EN ISO 1797-1 and DIN EN ISO 1797-2).

The rotational speeds recommended by manufacturers for driving dental tools can only be achieved by instruments running at high speed, such as high-speed contra angle handpieces and turbines. Force-fitting clamping elements are incorporated in these instruments, which clamping elements hold the tool in position in both the stationary state and the operating state in order to prevent unintentional release and therefore a risk to the patient, the practitioner or third parties.

The torques which occur at the cutting edge of the tool are absorbed solely by the force-fitting connection and the prevailing friction torques caused by the surface finishes. Form-fitting connections are also conceivable.

Depending on the selected design of the tool cutting edge, the crown substance to be processed, the geometry of the crown and the set rotational speed of the instrument, jerky, knocking and tugging impulses acting on the clamping element can occur during an operating procedure using the tool. Even at low peak loads, this can result in a sliding of the tool shaft or slippage in the clamping region and can damage the surface of the shaft.

In daily practice, the instructions of the tool and instrument manufacturers to only use smooth and undamaged shafts are often ignored and the tools are used until the cutting edge loses its function. This incurs the risk of a loss of quality of the instruments or the clamping systems. In practice, as known from market observations and customer surveys, this results in the following problems: overheating of instruments owing to the frictional heat generated by slippage, jamming tools or tools which can only be removed from the treatment instrument with increased effort, to a lack of, or an inadequate, holding force of the clamping element. In particular, this essentially affects the lifetime of the relevant treatment instruments, i.e. the contra angle handpieces, for example.

In the case of high-speed contra angle handpieces and turbines, a low and pleasant running noise, a low bur eccentricity and the product life span are important quality features which are influenced quite substantially by the quality of the individual components and assemblies. In particular, the quality of the rotating parts, such as the head drive or turbine rotor, has a significant effect on the quality of the overall product. As a result of high rotational frequencies of these assemblies, even small deviations in the form of components from the ideal state, such as unroundnesses, axial runouts, radial runouts or deviations with respect to the coaxiality can have a significant effect on the above-described quality features of the overall product.

With respect to the fastening of an inserted tool, high-speed contra angle handpieces and turbines generally possess a comparable structural design. This comprises a clamping element for holding the tool in position and at least one guide region for guiding the tool. Moreover, there is a usually axially movable element for enabling the clamping element to be operated using manual force. Serving to guide these parts, there is a bearing sleeve or drive sleeve or a so-called head drive for high-speed contra angle handpieces or an axle for turbines. To enable tools with smooth shafts to be guided and held in a permanently secure manner, components made from high-grade, hardenable stainless steels or stainless steels with a hard coating—often even made from hard metal—are incorporated in the clamping system, which components are already integrated in the bearing sleeve, pressed in and/or welded to one another. Ceramic materials are likewise used here.

DE 10 2004 007 413 A1 discloses a dentistry handpiece having a handpiece head in which a drivable receiving sleeve is rotatably mounted, into which a tool can be pushed by means of its shaft region. A toothed clutch acts between the receiving sleeve and the tool shaft to transmit a torque. Acting to prevent the tool shaft from sliding out of the receiving sleeve, there is an axial clutch having a ball as the clutch element, which, to this end, engages in a clutch hole formed on the tool shaft, thereby achieving spot-clamping between the ball and the tool shaft.

The invention is based on the object of providing a dental treatment instrument for operating a rotary tool, which has advantageous properties with respect to the disadvantages outlined above. In particular, the treatment instrument is intended to be suitable for meeting particularly high requirements such as those presented in the removal of crowns, for example.

This object is achieved according to the invention by the subject matter mentioned in the independent claim. Particular embodiments of the invention are described in the dependent claims.

According to the invention, a dental treatment instrument for operating a rotary tool is provided, wherein the treatment instrument has a head drive, which is provided for coupling to a shaft of the rotary tool and has a sleeve-like guide bushing. To fasten the rotary tool inserted into the guide bushing, the guide bushing has a collet chuck. In this case, the guide bushing has surface sections, at least in a first axial region, which form parts of a cylindrical form, wherein recesses are provided, which face radially outwards from these surface sections and give the inner contour of the guide bushing a form which deviates from a rotationally symmetrical form.

As a result of the surface sections, which form parts of a cylindrical form, particularly suitable guidance or support of a circular cylindrical shaft of a rotary tool can be realized. Moreover, the recesses are suitable for the engagement of radial projections of a non-circular cylindrical shaft section of a rotary tool so that a non-circular cylindrical shaft section of this type can be particularly suitably driven by a form-fitting contour. Therefore, the treatment instrument according to the invention offers, on the one hand, the option of receiving tool shafts with a correspondingly “altered” shaft geometry for meeting corresponding, particularly high requirements and counteracting the said disadvantages and weak points of a shaft according to DIN EN ISO 1797 and, on the other, the option of still being able to use the tools according to DIN EN ISO 1797 for standard applications.

In this case, the guide bushing preferably forms a second axial region whereof the inner contour forms a complete cylindrical form which, in terms of its diameter, is identical to the cylindrical form formed by the surface sections of the first axial region. This is advantageous with respect to the most suitable guidance of the circular cylindrical shaft along its longitudinal axis.

Sections of the second axial region preferably comprise the collet chuck. A purely cylindrical shaft of a rotary tool can thus be particularly suitably fastened in position by the collet chuck. In particular, to this end, the configuration can be such that the inner contour of the second axial region is partly formed by the collet chuck.

The first axial region preferably adjoins an entry opening for the tool. A rotary tool with a corresponding non-circular cylindrical shaft can thus be introduced particularly easily into the guide bushing, in particular when the shaft has radial engagement elements for engaging in the recesses, which engagement elements are fixedly connected to the rest of the shaft.

A length or the length of the first axial region is preferably shorter than a length or the length of the second axial region. Particularly suitable guidance of a tool shaft can thus be achieved; for example it can be provided that the length of the first axial region is, at most, 20% of the length of the second axial region.

The guide bushing preferably has a bearing sleeve or drive sleeve, which is rotatably mounted in a head region of the treatment instrument and cooperates with drive means of the treatment instrument. A particularly effective torque transmission to the shaft of the tool can thus be realized. In this case, the first axial region of the guide bushing is furthermore preferably configured as an integral part of the drive sleeve. A particularly direct torque transmission is thus enabled.

The drive sleeve preferably consists of a harder substance than the collet chuck. This is advantageous with respect to the suitability of the drive sleeve for the torque transmission and the suitability of the collet chuck for fastening the rotary tool.

The recesses preferably form elongated notches which extend axially. In this case, a plurality of notches are furthermore preferably provided, which are arranged distributed, preferably evenly distributed, on the circumference of the rotational form. This is advantageous in manufacturing terms and, with this, enables a suitable torque transmission.

The treatment instrument preferably has drive means for transmitting a rotation to a rotary tool received in the guide bushing. In particular, these drive means can comprise an electric motor or a turbine or an air supply/compressed air. The drive means can be of an electrical or fluid type. For example, the drive means can comprise an air motor.

The collet chuck is preferably inserted into a radial recess of the drive sleeve. In a suitable manufacturing option, this is advantageous with respect to the torque transmission from the drive sleeve to the collet chuck and subsequently to the tool.

With respect to the longitudinal axis, the collet chuck is preferably arranged on a side of the first axial region which is opposite the entry opening. The guide bushing is thus suitable for fastening a tool which has a cylindrical shaft section which is adjoined by a further shaft section which is configured for form-fitting interaction with the first axial region.

The first axial region of the guide bushing is preferably configured with the help of a separate component which is inserted into the drive sleeve. This is advantageous in manufacturing terms. In this case, with respect to the longitudinal axis, the collet chuck is furthermore preferably arranged on a side of the separate component which is opposite the entry opening. With respect to the fastening option for a tool with a cylindrical shaft section and a further shaft section adjoining this, this is in turn advantageous for the form-fitting interaction with the first axial region.

The invention will be explained in more detail below with the aid of an exemplary embodiment and with respect to the drawings, which show:

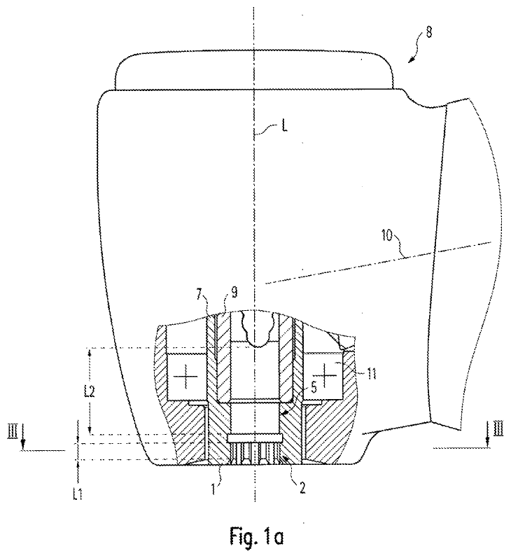

FIG. 1a a schematic sketch of a side view of a head region of a treatment instrument according to the application, wherein a region of a guide bushing, which serves for receiving a tool shaft, is shown as a cross-section.

FIG. 1b a cross-sectional sketch corresponding to FIG. 1a,

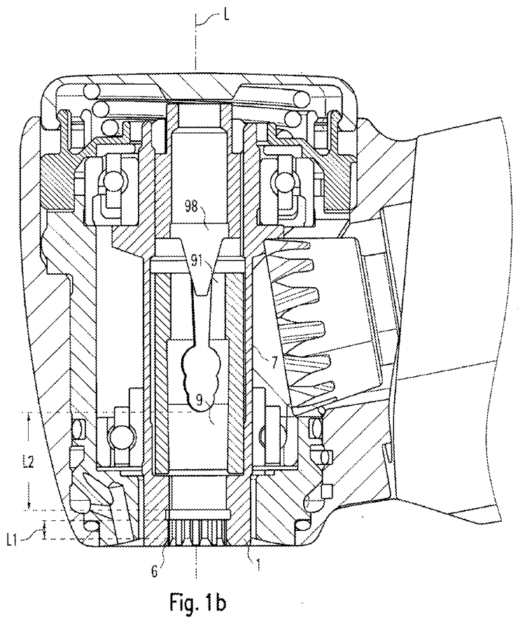

FIG. 2 an enlarged detail of FIG. 1a,

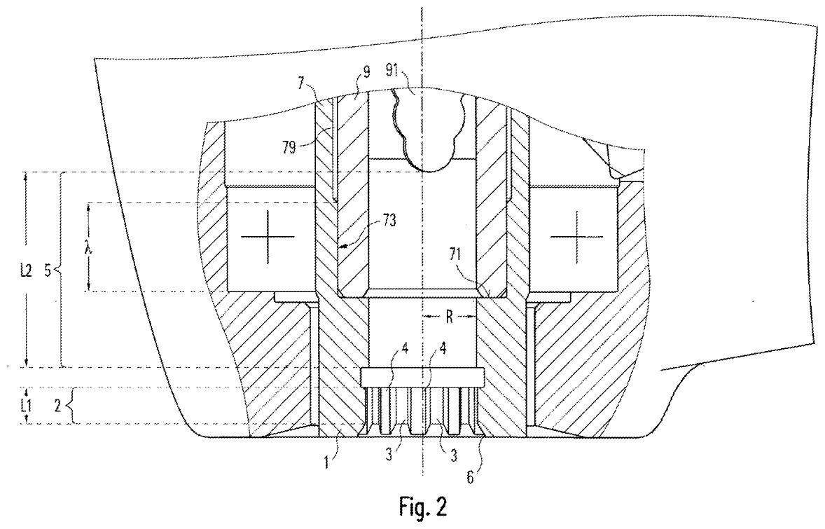

FIG. 3 a sketch of a section of the guide bushing along the line indicated in FIG. 1a,

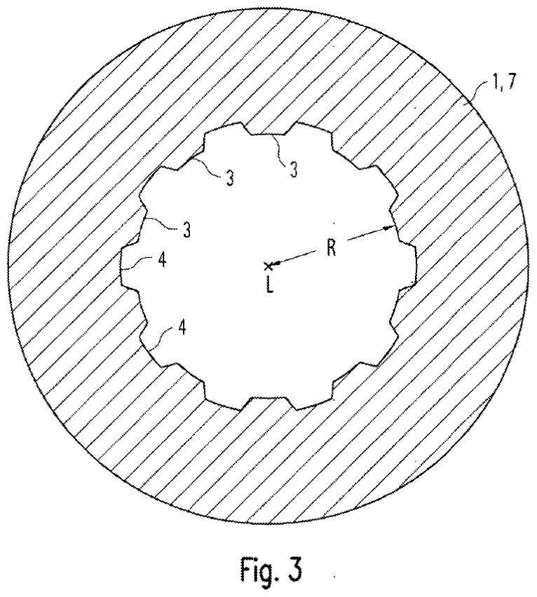

FIG. 4a an illustration, corresponding to the illustration of FIG. 1a, of a variant in which a first axial region of the guide bushing is configured as an integral part of a collet chuck,

FIG. 4b a cross-sectional sketch corresponding to FIG. 4a,

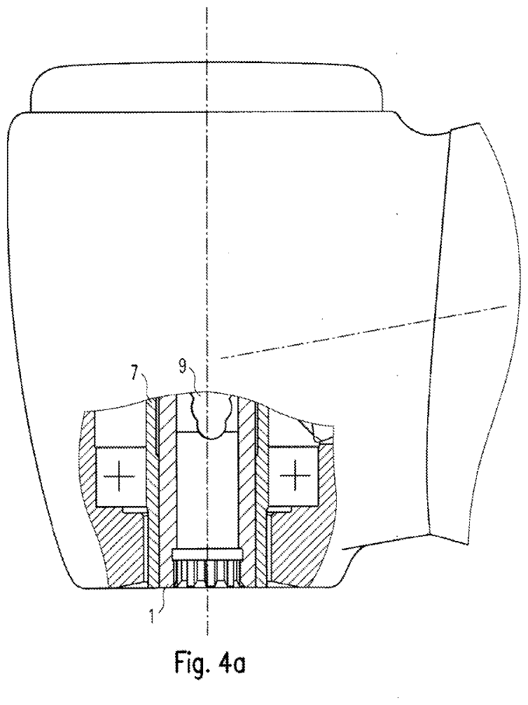

FIG. 5 an illustration, corresponding to the illustration of FIG. 4a, of a further variant in which the first axial region of the guide bushing is configured with the help of an independent component,

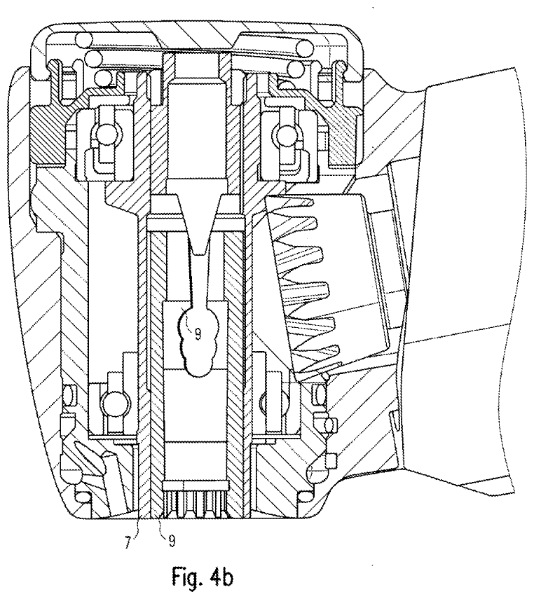

FIG. 6 a perspective sectional sketch of the component shown in FIG. 5,

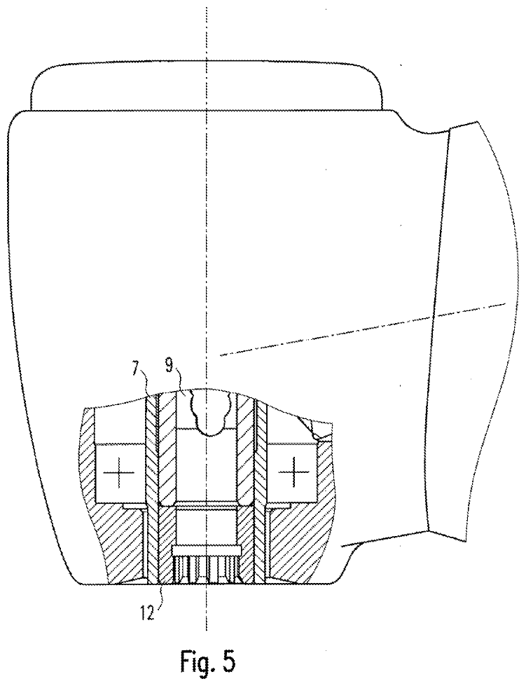

FIG. 7 a view of the component shown in FIG. 5 along the longitudinal axis,

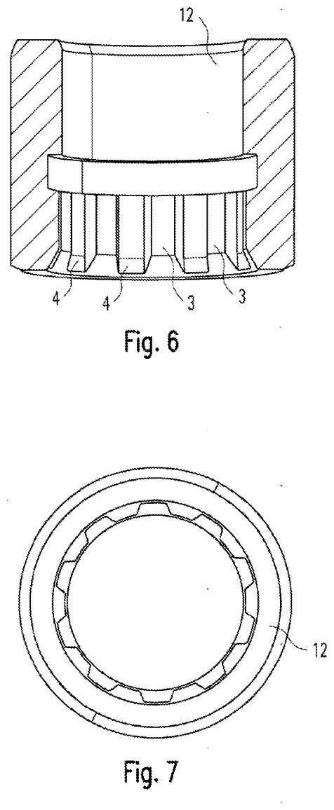

FIG. 8 a perspective sectional sketch of a variation of the component shown in FIG. 5 and

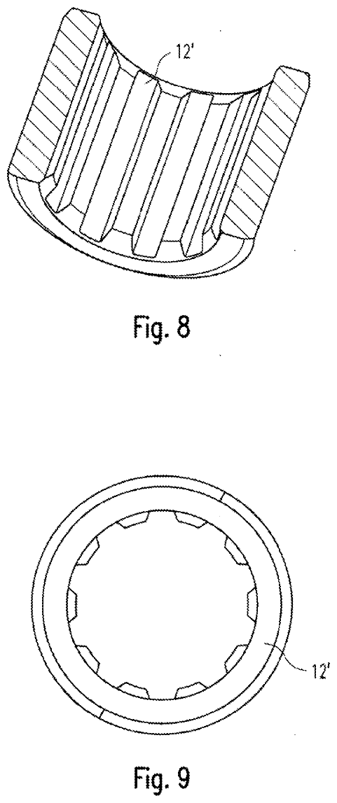

FIG. 9 a view of the variation of the component shown in FIG. 8 along the longitudinal axis.

FIG. 1a shows a schematic sketch of a side view of a head region 8 of a dental treatment instrument according to the application for operating a rotary tool. FIG. 1b shows a corresponding cross-sectional sketch. The treatment instrument can be, for example, a contra angle handpiece, in particular a high-speed contra angle handpiece, or a turbine.

The rotary tool, also referred to below as tool for short, can be, for example, a bur. In particular, the tool has a shaft region, or shaft for short, which is provided to be pushed into the head region 8 of the treatment instrument.

As is known per se and conventional, the treatment instrument can have a handle part, which adjoins the head region 8 and is provided for holding the treatment instrument. Only a main axis 10 of the handle part is indicated in FIG. 1a.

The treatment instrument has a head drive, which is provided for coupling to the shaft of the tool, is arranged in particular in the head region 8 and has a sleeve-like guide bushing 1. In particular, the guide bushing 1 is configured such that the shaft of the tool can be pushed into the guide bushing 1 for coupling purposes. A longitudinal axis L is specified through the guide bushing 1. The configuration in this case is such that the shaft of the tool is aligned with its shaft axis parallel to the longitudinal axis L when the tool is connected to the treatment instrument as intended, i.e. inserted into the guide bushing 1.

As sketched in FIG. 1b, the guide bushing 1 has a collet chuck 9 for fastening a rotary tool received in the guide bushing 1. In particular, the collet chuck 9 is configured both for securing the tool axially and also fastening it such that the transmission of the torque from the guide bushing 1 to the tool is suitably enabled for operation of the treatment instrument.

In particular, the configuration is such that the guide bushing 1 is mounted in the head region 8 of the treatment instrument such that it is rotatable, for example rotatable by means of a rotary bearing 11, and is configured for transmitting a torque to the shaft of the tool in order to thereby drive the tool. To this end, the treatment instrument preferably has drive means for transmitting the torque or a rotation to the tool received in the guide bushing 1. In this case, the drive means can comprise, in particular, an electric motor or a turbine.

FIG. 2 shows an enlarged detail of FIG. 1a and, in FIG. 3, a cross-sectional sketch of the guide bushing 1 is shown normal to the longitudinal axis L and, more precisely, at the level indicated by in FIG. 1a. As seen along the longitudinal axis L, the guide bushing has, at least in a first longitudinal section 2, indicated here as a first axial region 2, surface sections 3 which form parts of a cylindrical form, in particular a circular cylindrical form, which extends with its axis of symmetry parallel to the longitudinal axis L. In FIG. 3, the reference sign L indicates the position of the longitudinal axis and the radius of the said cylindrical form is indicated by the reference sign R. Accordingly, the diameter of the cylindrical form is 2R. The configuration is preferably such that—as seen in a cross-section normal to the longitudinal axis L—the surface sections 3 each describe an arc whereof the central angle is at least 5°, particularly preferably at least 10°. This is advantageous because a tool with a purely cylindrical shaft section, whereof the radius is R, can thus be particularly suitably radially secured by the surface sections 3.

As is furthermore sketched by way of example in FIG. 3, the surface sections 3 are formed such that they are preferably evenly distributed with respect to the circumference of the cylindrical form. In the example shown, the number of surface sections 3 is ten. The number can be, for example, between two and thirty, preferably between four and twenty.

The configuration is preferably such that the surface sections 3 each have an elongated form, wherein, in particular, they each extend parallel to the longitudinal axis L.

From the surface sections 3, recesses 4 are provided or formed which, with respect to the longitudinal axis L, face radially outwards and which give the inner contour of the guide bushing 1 a form which deviates from a rotationally symmetrical or circular cylindrical form.

A tool with a shaft which has radial engagement elements which correspond to the recesses 4 is therefore particularly suitable for the torque transmission since, in this case, a corresponding form-fitting connection can be established between the tool and the guide bushing 1. Using a tool of this type, the treatment instrument is therefore in particular suitable for transmitting particularly high torques, such as those which can typically occur in the course of removing a crown, for example. In particular, the guide bushing 1 is suitable for fastening a tool with a shaft which has a purely circular cylindrical form in a first shaft region, in particular with the radius R, and has the said radial engagement elements in a second region, the radial extent of which engagement elements is correspondingly greater than R.

However, the treatment instrument is not only suitable for operating a tool with a shaft which has corresponding engagement elements, but alternatively also for operating a rotary tool with a purely cylindrical shaft section or a shaft according to the standard DIN EN ISO 1797 mentioned at the outset, since such a purely cylindrical shaft or shaft section can be correspondingly suitably fastened by the collet chuck.

The configuration is preferably such that the recesses 4 each have an elongated form, wherein they each extend in particular parallel to the longitudinal axis L. For example, the recesses 4 can form elongated notches or be formed by elongated notches. In this case, a plurality of notches are preferably provided, which are arranged distributed, preferably evenly distributed, on the circumference of the rotational form. The number of recesses 4 is preferably equal to the number of surface sections 3.

In this case, the form-fitting contour can be designed for example as a key shaft connection or profile shaft connection, such as a disk connection, key connection or splined shaft connection or toothed shaft connection, for example. Form-fitting connections are releasable connections and differ in terms of their areas of application. In the event of high notch stresses, key or splined shaft connections with parallel flanks are preferred. However, key connections are disadvantageous in that an unsuitable imbalance can occur at high rotational speeds. Toothed shaft connections or serrated shaft connections, which are suitable inter alia for low profile heights, lend themselves to low notch stresses. In manufacturing terms, they can be advantageously produced by stamping, for example. Any peak loads which occur at the form-fitting connection are distributed to the gear components and could possibly be equalized via a slip clutch.

In manufacturing terms, the described inner contour of the guide bushing 1 can be advantageously incorporated by broaching, splicing or stamping, for example.

The configuration is preferably such that, as seen over the first axial region 2, the inner contour of the guide bushing 1 has no part which has a spacing from the longitudinal axis L smaller than the radius R of the cylindrical form. In particular, the configuration is such that, as seen over the first axial region 2, the inner contour of the guide bushing 1 is formed merely by the surface sections 3 and the recesses 4. The guide bushing 1 is thus particularly suitable for receiving a circular cylindrical tool shaft with the diameter 2R. The diameter 2R is preferably selected such that the guide bushing 1 is suitable for receiving a tool shaft which corresponds to the standard DIN EN ISO 1797 mentioned at the outset.

As is the case in the example shown and indicated in FIG. 1a, the guide bushing 1, as moreover viewed along the longitudinal axis L, has a second longitudinal section 5, indicated here as a second axial region 5, whereof the inner contour forms a complete cylindrical form. In this case, the diameter of this complete cylindrical form is equal to the diameter 2R of the cylindrical form formed by the surface sections 3 of the first axial region 2. Therefore, this second axial region 5 is particularly suitable for guiding a corresponding shaft.

In particular, the guide bushing 1 can be configured such that sections of the second axial region 5 comprise the collet chuck 9.

The collet chuck 9 can be part of the second axial region 5. The inner contour of the second axial region 5 is preferably partly, preferably at least mostly, formed by the collet chuck 9. As revealed by FIGS. 1a and 2, in the exemplary embodiment shown therein the inner contour of the second axial region 5 is predominantly or mostly formed by the collet chuck 9.

The second axial region 5 can directly adjoin the first axial region 2. Alternatively—as sketched by way of example in FIGS. 1a and 2—between the first axial region 2 and the second axial region 5, as seen along the longitudinal axis L, an intermediate section can be provided, in which, in the radial direction, the inner contour of the guide bushing 1 is continuously further away from the longitudinal axis L than the radius R.

The first axial region 2 preferably adjoins an entry opening 6 for the tool, in particular directly. In this case, the entry opening 6 is preferably configured as part of the head region 8, in particular through the guide bushing 1. With respect to the longitudinal axis L, the second axial region 5 preferably extends on a side of the first axial region 2 which is opposite the entry opening 6. With respect to the illustrations of FIGS. 1a and 2, the second axial region 5, in this sense, extends above the first axial region 2 and the entry opening 6 is formed on the underside of the first axial region 2.

The extent of the first axial region 2 along the longitudinal axis L—also identified here as the length L1 of the first axial region 2—is preferably shorter than the extent of the second axial region 5 along the longitudinal axis L—also identified here as the length L2 of the second axial region 5. For example, it can be provided that the length L1 of the first axial region 2 is, at most, 20% of the length L2 of the second axial region 5. This is advantageous with respect to a suitable guidance of the tool shaft.

As shown by way of example in FIG. 1b, the collet chuck 9 furthermore preferably comprises an end section 91 in which at least one gap is formed, which extends substantially along the longitudinal axis L. The treatment instrument furthermore preferably has a wedge-like engagement element 98, which is movably mounted parallel to the longitudinal axis L in the head region 8 such that it can engage to a greater or lesser extent in the at least one gap. In this case, the collet chuck 9 is configured to be resilient, such that a radial diameter of the inner contour of the end section 91 can be reduced or increased by a back and forth movement of the engagement element 98. Thus, by reducing the diameter of the inner contour of the end section 91 of the collet chuck 9, the said securing of a purely cylindrical shaft section of a tool which is inserted into the guide bushing 1 can be achieved so that the tool is arranged suitably fastened in the guide bushing 1 for operation. By increasing the diameter, the secured state can be released so that, after use, the tool can thus be simply, in particularly manually, removed from the guide bushing 1 again.

With respect to the longitudinal axis L, the end section 91 of the collet chuck 9 preferably extends on a side of the second axial section 5 which is opposite the first axial section 2. With respect to the illustration of FIG. 1b, the end section 91 of the collet chuck 9, in this sense, extends above the second axial section 5.

Moreover, the guide bushing 1 furthermore preferably has a bearing sleeve or drive sleeve 7, which is rotatably mounted in the head region 8, wherein the drive sleeve 7 cooperates with the drive means of the treatment instrument so that a particularly direct torque transmission is enabled. In this case, in the exemplary embodiment shown in FIG. 1a, the first axial region 2 of the guide bushing 1 is configured as an integral part of the drive sleeve 7. It can thus be achieved that the torque is transmitted particularly directly to the shaft of the tool.

With respect to the longitudinal axis L, the collet chuck 9 is preferably arranged on a side of the first axial region 2 which is opposite the entry opening 6.

With respect to the illustration of FIG. 1b, the collet chuck 9, in this sense, is arranged above the first axial region 2, whilst the entry opening 6 is formed on the underside of the first axial region 2.

The drive sleeve 7 preferably consists of a harder substance than the collet chuck 9. In other words, the collet chuck 9 preferably consists of a softer substance than the drive sleeve 7. This is advantageous because the collet chuck 9 is thus particularly suitable for fastening the tool.

In the configuration sketched in FIGS. 1a and 1b, the collet chuck 9 is configured as a separate component which is inserted, in particular fixedly and permanently inserted, for example pressed, into the drive sleeve 7 or into the rest of the guide bushing 1. As revealed for example by FIG. 1b, the guide bushing 1 can consist of the drive sleeve 7 and the collet chuck 9.

For example, as revealed by FIG. 1a, the second axial region 5 can be formed in part by the drive sleeve 7 and in further part by the collect chuck 9. With respect to FIG. 2, a lower portion of the second axial region 5 is formed only by the drive sleeve 7 and an upper portion of the second axial region 5 is formed radially inwardly by the collet chuck 9 and radially outwardly by the drive sleeve 7.

As mentioned, the collect chuck 9 can be inserted, for example pressed, into the drive sleeve 7. In particular, to this end, the drive sleeve 7 can have a radial recess 73 into which the collet chuck 9 is inserted accordingly.

The configuration here is furthermore preferably such that the drive sleeve 7 has a shoulder surface 71 which extends in particular in a plane which is orientated normally to the longitudinal axis L, wherein this shoulder surface 71 forms a bearing surface for the collet chuck 9.

As is furthermore revealed by FIGS. 1b and 2, the configuration in this case is preferably such that, with respect to the longitudinal axis L within a longitudinal section λ, the recess 73 forms an in particular circular cylindrical contact surface for direct contact with the collect chuck 9 such that, during operation, the torque transmission from the drive sleeve 7 to the collet chuck 9 takes place via this contact surface. For this—as sketched in FIG. 2—the longitudinal section λ is preferably located within the second axial region 5.

In this case, the configuration is furthermore preferably such that there is no provision for the separation of the collet chuck 9 from the drive sleeve 7 in the course of the intended operational use of the treatment instrument, i.e. this is not possible without damage, for example.

A radial gap 79 is preferably formed between the end section 91 of the collet chuck 9 and the drive sleeve 7. This gap is advantageous with respect to the described movement of the end section 91 of the collet chuck 9 when the diameter of the inner contour of the end section 91 is increased.

In FIG. 4a, a variant of the configuration according to FIG. 1a is shown, wherein the reference signs are used analogously to above. FIG. 4b shows a corresponding cross-sectional sketch. In this configuration, the inner contour of the first axial region 2 is formed completely by the collet chuck 9. Accordingly, in particular the surface sections 3 and the recesses 4 are formed completely by the collet chuck 9. In other words, the first axial region 2 is formed radially inwardly by the collet chuck 9 here and radially outwardly by the drive sleeve 7.

In this case, the configuration is preferably such that the collet chuck 9 is fixedly connected to the drive sleeve 7, in particular such that there is no provision for the separation of the collet chuck 9 from the drive sleeve 7 in the course of the intended operational use of the treatment instrument, i.e. this is not possible without damage. Therefore, a particularly suitable torque transmission between the drive sleeve 7 and the collet chuck 9 can be particularly suitably achieved.

FIG. 5 shows a further variation. The first axial region 2 of the guide bushing 1 is configured with the help of a separate component 12 here, which, like the collet chuck 9 in the last-mentioned example, is correspondingly fixedly inserted into the drive sleeve 7. The first axial region 2 is formed radially inwardly by the component 12 here and radially outwardly by the drive sleeve 7.

As is furthermore revealed by FIG. 5, with respect to the longitudinal axis L, the collet chuck 9 here is arranged on a side of the separate component 12 which is opposite the entry opening 6. With respect to the illustration of FIG. 5, the collet chuck 9, in this sense, is arranged above the component 12.

FIG. 6 shows a perspective sectional sketch of the component 12 shown in FIG. 5 and FIG. 7 shows a view of the component 12 along the longitudinal axis L. In this case, the configuration can be such that the recesses 4—as sketched—extend merely over a sub-region of the longitudinal extent of the component 12 along the longitudinal axis L. Over a further sub-region of its longitudinal extent, the component 12, analogously to above, has a completely circular cylindrical inner contour with the radius R. This further sub-region in turn serves accordingly for guiding a correspondingly cylindrical tool shaft.

Between the first-mentioned sub-region with the recesses 4 the last-mentioned further sub-region with the circular cylindrical inner contour, a second section can be configured whereof the delimiting walls, as seen in the radial direction, are continuously further away from the longitudinal axis L than the radius R.

FIG. 8 shows a perspective sectional sketch of a variation to the component shown in FIG. 5, denoted by the reference sign 12′ here, and FIG. 9 shows a view of this component 12′ along the longitudinal axis. In this configuration, the structure formed on the inside is configured continuously, as it were, i.e. it extends over the entire longitudinal extent of the component 12′.

Moreover, the clamping section can be configured in a manner known per se. For example, to release the tool or for a tool change, a corresponding push-button actuation, which is known per se, can be provided.

The drive sleeve 7 can consist of a hardenable stainless steel, in particular with a hardness grade greater than 450 HV. The collet chuck 9 can consist of a hardenable stainless steel. The collet chuck 9 can be configured such that it has a clamping force of greater than 22 N. The component 12 can consist of a hardenable stainless steel, for example, or a hard metal, for example.

### With a Treatment Instrument According to the Application, the Following Advantages, in Particular, can be Achieved:

Torques which occur can be absorbed and slippage can therefore be prevented.
By preventing slippage, additional heating caused by friction at the form-fitting connection can, in particular, be prevented.
The clamping system is protected from damage and can therefore ensure reliable functioning.
The tool shafts are protected from damage and the problem of jamming tools or tools which can only be removed with difficulty can be practically prevented.
The treatment instrument can be advantageously configured as a high-speed contra angle handpiece or as a turbine.
For the clamping system, a particularly long useful life can be achieved, in particular in comparison to clamping systems of comparable series instruments.
Many standard components of a known head housing can still be used.
Despite the option of the form-fitting connection, standard tools according to DIN EN ISO 1797 can alternatively still be used.
In this case, a customary rapid and (virtually) positionally independent insertion of the tool into the clamping system is furthermore enabled.
A tool change is possible via the customary push button.
The tool can be safely and tightly guided in the drive sleeve or the guide bushing; this is advantageous with respect to a low bur eccentricity and a suitable smoothness.

1. A dental treatment instrument for operating a rotary tool, wherein the treatment instrument has a head drive, which is provided for coupling to a shaft of the rotary tool and has a sleeve-like guide bushing,
wherein the guide bushing has a collet chuck for fastening the rotary tool inserted into the guide bushing,
and wherein the guide bushing has surface sections, at least in a first axial region, which form parts of a cylindrical form, wherein recesses are provided, which face radially outwards from these surface sections and give the inner contour of the guide bushing a form which deviates from a rotationally symmetrical form.

2. The dental treatment instrument as claimed in claim 1,
characterized in that
the guide bushing has a second axial region, whereof the inner contour forms a complete cylindrical form which, in terms of its diameter, is identical to the cylindrical form formed by the surface sections of the first axial region.

3. The dental treatment instrument as claimed in claim 2,
characterized in that
sections of the second axial region comprise the collet chuck.

4. The dental treatment instrument as claimed in claim 2, characterized in that the inner contour of the second axial region is partly formed by the collet chuck.

5. The dental treatment instrument as claimed in claim 2,
characterized in that
the first axial region adjoins an entry opening for the tool.

6. The dental treatment instrument as claimed in claim 2,
characterized in that
the length of the first axial region is shorter than the length of the second axial region.

7. The dental treatment instrument as claimed in claim 6,
characterized in that
the length of the first axial region is, at most, 20% of the length of the second axial region.

8. The dental treatment instrument as claimed in claim 1,
characterized in that
the guide bushing has a bearing sleeve or drive sleeve, which is rotatably mounted in a head region of the treatment instrument and cooperates with drive means of the treatment instrument.

9. The dental treatment instrument as claimed in claim 8,
characterized in that
the first axial region of the guide bushing is configured as a integral part of the drive sleeve.

10. The dental treatment instrument as claimed in claim 8,
characterized in that
the drive sleeve consists of a harder substance than the collet chuck.

11. The dental treatment instrument as claimed in claim 1,
characterized in that the recesses form elongated notches which extend axially.

12. The dental treatment instrument as claimed in claim 11,
characterized in that a plurality of notches are provided, which are arranged distributed, preferably evenly distributed, on the circumference of the rotational form.

13. The dental treatment instrument as claimed in claim 1,
characterized in that
it has drive means for transmitting a rotation to a rotary tool received in the guide bushing.

14. The dental treatment instrument as claimed in claim 13,
characterized in that
the drive means comprise an electric motor.

15. The dental treatment instrument as claimed in claim 13,
characterized in that
the drive means comprise a turbine.

16. The dental treatment instrument as claimed in claim 8,
characterized in that
the collet chuck is inserted into a radial recess of the drive sleeve.

17. (canceled)

18. The dental treatment instrument as claimed in claim 8,
characterized in that
the first axial region of the guide bushing is configured with the help of a separate component which is inserted in the drive sleeve.

19. (canceled)
